(19) United States
(12) Patent Application Publication
(10) Pub. No.: US 2025/0127176 A1
(43) Pub. Date: Apr. 24, 2025

(54) BIO-FUNGICIDES FORMULATIONS FOR INHIBITING PHYTOPHTHORA INFESTANS AND METHOD THEREOF

(71) Applicants: COROMANDEL INTERNATIONAL LIMITED, Secunderabad (IN); TAMIL NADU AGRICULTURAL UNIVERSITY, Coimbatore (IN)

(72) Inventors: Baburaj LAKSHMI KANTHAN, Secunderabad (IN); Balaji SAMBAMOORTHY, Secunderabad (IN); Balakumar CHINNASAMY, Secunderabad (IN); Karthic CHINNUSAMY, Secunderabad (IN); Vignesh MURTHY, Secunderabad (IN); Gokila SPIRAMAN, Secunderabad (IN); Mahendra KADIRI, Coimbatore (IN); Nakkeeran SEVUGAPPERUMAL, Coimbatore (IN); Arul LOGANATHAN, Coimbatore (IN); Sivakumar UTHANDI, Coimbatore (IN)

(21) Appl. No.: 18/695,879

(22) PCT Filed: Sep. 30, 2022
(86) PCT No.: PCT/IN2022/050877

§ 371 Date: Mar. 27, 2024

(30) Foreign Application Priority Data
Oct. 1, 2021 (IN) 202141020069

(51) Int. Cl. A01N 63/22; A01N 25/24; A01N 25/30; A01P 3/00; C12N 1/20; C12R 1/07; C12R 1/125

(52) U.S. Cl. CPC A01N 63/22; A01N 25/24; A01N 25/30; A01P 3/00; C12N 1/205; C12R 2001/07; C12R 2001/125

(57) ABSTRACT

The present invention relates to bio-fungicides that inhibits Phytophthora infestans. More particularly, the present invention relates to novel strains of Bacillus spp. that can inhibit Phytophthora infestans. The present invention also relates to bio-fungicidal formulations comprising novel strains of Bacillus spp. either alone or a mixture of novel strains of Bacillus spp., in combination with other excipients.

### FIELD OF THE INVENTION

The present invention relates to bio-fungicides that inhibits Phytophthora infestans. More particularly, the present invention relates to novel strains of Bacillus spp. that can inhibit Phytophthora infestans. The present invention also relates to bio-fungicidal formulations comprising novel strains of Bacillus spp. either alone or a mixture of novel strains of Bacillus spp., in combination with other excipients.

### BACKGROUND OF THE INVENTION

Late blight is caused by oomycete fungal pathogen Phytophthora infestans. This potentially devastating pathogen can infect potato foliage and tubers at any stage of crop growth and development. The primary host is potato, but P. infestans can also infect other solanaceous plants, including tomatoes, petunias and hairy nightshade, that can act as source of inoculum for causing late blight of potato. Late blight of potato and tomato were the diseases that was responsible for the Irish potato famine in the mid-nineteenth century. P. infestans can infect and destroy the leaves, stems, fruits, and tubers of potato and tomato plants. Before the disease appeared in Ireland it caused a devastating epidemic in the early 1840s in the north-eastern United States.

The pathogen P. infestans is favoured by moist, cool environments. Formation of sporangia and zoospore is optimal at 12-18° C. (54-64° F.) in water-saturated or nearly saturated environments, and zoospore production is favoured at temperatures below 15° C. (59° F.). Lesion growth rates are typically optimal at a slightly warmer temperature range of 20 to 24° C. (68 to 75° F.).

Phytophthora spp. is one type of plant pest that can cause severe economic losses in the agricultural and horticultural industries. Chemical agents can be used to control fungal phytopathogens, but the use of chemical agents suffers from disadvantages including high cost, lack of efficacy, emergence of resistant strains of the fungi, and undesirable environmental impacts. In addition, such chemical treatments tend to be in discriminant and may adversely affect beneficial bacteria, fungi and arthropods in addition to the plant pathogen at which the treatments are targeted.

Natural products are substances produced by microbes, plants, and other organisms. Natural product pesticides have a potential to play an important role in controlling pests in both conventional and organic farms. Secondary metabolites produced by microbes (bacteria, actinomycetes and fungi) provide novel chemical compounds which can be used either alone or in combination with known compounds like pesticides to effectively control pathogenic fungi and to reduce the risk for resistance development. There are several well-known examples of microbial natural products that are successful as agricultural fungicides.

The development of a microbial fungicide starts with the isolation of a microbe in a pure culture. It then proceeds with efficacy and spectrum screening using in vitro, in vivo or pilot scale trials in a greenhouse and in the field. At the same time, active compounds produced by the microbe are isolated and identified. For the commercialization of a microbial fungicide, the microbe has to be economically produced by fermentation at an industrial scale and formulated with approved biocompatible additives to increase efficacy and to maximize the ease of application.

With the development of increasing resistance to chemical fungicides like Captan, folpet, dithiocarbamates, pentachlorophenol, and mercurial, the spectrum of available fungicides is narrowing. In addition, non-naturally occurring fungicides can have detrimental environmental effects.

Hence, there is a need in the art to develop naturally occurring fungicides to which plant pathogens have not developed resistance, and which have minimal environmental effects.

### OBJECT OF THE INVENTION

An object of the present invention is to provide an effective microbial isolate to control late blight of potato.

Another object of the present invention is to provide an effective microbial isolate of Bacillus spp.

Another object of the present invention is to provide bio fungicidal formulations of Bacillus spp.

Another object of the present invention is to provide viable bio fungicidal formulations during shelf life.

### SUMMARY OF THE INVENTION

The present invention provides a bio-fungicide formulations comprising a biologically pure culture of Bacillus spp. and excipients, wherein the Bacillus spp. is selected from the group comprising of Bacillus amyloliquefaciens—MTCC 25554, Bacillus subtilis subsp. spizizenii—MTCC 25552, Bacillus amyloliquefaciens—MTCC 25555, Bacillus velezensis—MTCC 25553, Bacillus safensis subsp. safensis—MTCC 25556 or combination thereof, wherein the Bacillus spp. is present in a range from 1 to 98% (wt./wt.).

In an embodiment of the present invention, the excipient is selected from surfactant, encapsulant, adjuvant, sticking agents, preservative and water.

In another embodiment of the present invention, the surfactant is selected from the group comprising of Polyethylene glycol (PEG)-100, PEG-200, PEG-300, PEG-400, PEG-8000, PEG-20000, Tween-20, Tween-40, Tween-60, Tween-80, Span-20, Span-40, PEG 6000, glycerol, PVP K30, Tween 85 or combination thereof present in a range from 0.001 to 31% (wt./wt.).

In another embodiment of the present invention, the encapsulant is selected from the group comprising glycerol, PEG (Polyethylene glycol) and PVP (Polyvinylpyrrolidone), preferably, glycerol present in a range from 2 to 5% (wt./wt.).

In another embodiment of the present invention, the sticking agent is xanthan gum, 0.4% xanthan gum, guar gum, gum acacia, Arabic gum, malto dextrin, starch soluble and insoluble, carboxy methyl cellulose or combination thereof, preferably, the sticking agent is xanthan gum present in a range from 0.05 to 82% (wt./wt.).

In another embodiment of the present invention, the adjuvant is selected from the group comprising of olive oil, cashew oil, castor oil, sunflower oil, Pongamia oil, sesame oil, linseed oil, rice bran oil, ground nut oil or combination thereof present in a range from 2 to 98% (wt./wt.) preferably, the adjuvant is linseed oil and sesame oil.

In another embodiment of the present invention, the preservative is selected from the group comprising of citric acid, calcium propionate, copper sulphate, magnesium sulphate, boric acid, calcium benzoate, molybdenum or combination thereof present in a range from 0.01 to 0.25% (wt./wt.).

In another embodiment of the present invention, the formulation is formulated as an aqueous solution or emulsifiable concentrate.

In another embodiment of the present invention, the fungal infections are caused by Phytophthora infestans.

In another embodiment of the present invention, the formulation optionally comprises a chemical or biological pesticide.

### BRIEF DESCRIPTION OF THE FIGURES

FIG. 1 is Genome of B. safensis subsp. safensis—MTCC 25556

FIG. 2 shows Phylogenetic placement of B. safensis subsp. safensis—MTCC 25556

FIG. 3 is Genome of B. amyloliquefaciens—MTCC 25554

FIG. 4 shows Phylogenetic placement of B. amyloliquefaciens—MTCC 25554

FIG. 5 is Genome of B. subtilis subsp. spizizenii—MTCC 25552

FIG. 6 shows Phylogenetic placement of B. subtilis subsp. spizizenii—MTCC 25552

FIG. 7 is Genome of B. amyloliquefaciens—MTCC 25555

FIG. 8 shows Phylogenetic placement of B. amyloliquefaciens—MTCC 25555

FIG. 9 is Genome of B. velezensis—MTCC 25553

FIG. 10 shows Phylogenetic placement of B. velezensis—MTCC 25553

FIG. 11 shows Ultramicroscopic changes of P. infestans observed under SEM; S: Sporangia, H: Hypha, arrow indicates bacterial colonization: B. safensis subsp. safensis—MTCC 25556 (1&2), B. amyloliquefaciens—MTCC 25554 (3&4), B. subtilis subsp. spizizenii—MTCC 25552 (5&6), B. velezensis—MTCC 25553 (7&8), B. amyloliquefaciens—MTCC 25555 (9&10) and Healthy mycelium (11&12)

FIG. 12 shows Ultramicroscopic changes on colonization potential and antagonistic activity of bacterial endophytes against P. infestans observed under SEM; S: Sporangia, H: Hyphal colonization, arrow indicates bacterial colonization (except in control). 1-3: Inoculated control (P. infestans on potato leaf), 4-6: B. safensis—MTCC 25556 and P. infestans in potato leaf, 7-9: B. amyloliquefaciens—MTCC 25554 and P. infestans in potato leaf, 10-12: B. subtilis subsp. Spizizenii—MTCC 25552 and P. infestans on potato leaf, 13-15: B. velezensis—MTCC 25553 and P. infestans on potato leaf, 16-19: B. amyloliquefaciens—MTCC 25555 and P. infestans on potato leaf.

FIG. 13 shows comparative efficacy of antagonistic activity of effective bacterial endophytes with competitive products.

### DESCRIPTION OF THE INVENTION

The present invention relates to a novel strains of Bacillus spp. having antagonistic activity against Phytophthora infestans when used alone or in combination with other strains of Bacillus spp. The present invention also relates to method of preparation of formulations of novel strains of Bacillus spp. either alone, or in combination with other strains of Bacillus spp. and with excipients.

It is to be noted, as used in the specification and claims, the singular forms “a”, “an”, and “the” include plural referents unless the context clearly dictates otherwise. Thus, for example, reference to a formulation containing “a compound includes a mixture of two or more compounds. It should also be noted that the term “or” is generally employed in its sense including “and/or” unless the content clearly dictates otherwise. The expression of various quantities in the terms of “% w/w” or “%” means the percentage by weight, relative to the weight of the total formulation unless otherwise specified.

Similarly, the words “comprise,” “comprises,” and “comprising” are to be interpreted inclusively rather than exclusively. Likewise, the terms “include” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of and “consisting of the disclosed components. Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

As used herein, “biological control” is defined as controlling of a pathogen by using its antagonist.

As used herein the term “fungus’ or “fungi’ includes a wide variety of nucleated, spore bearing organisms which are devoid of chlorophyll. Examples of fungi include yeasts, mildews, moulds, rusts and mushrooms.

As used herein the term “bacteria” are prokaryotic organisms that does not have a distinct nucleus.

As used herein the term “fungicide” means a substance which has an ability to increase mortality or to inhibit growth rate of fungi.

As used herein the term “culture” refers to the propagation of organisms on or in various kinds of media.

As defined herein, “derived from” means directly isolated or obtained from a particular source or alternatively having identifying characteristics of a substance or organism isolated or obtained from a particular source. If the “source” is an organism, “derived from” means that it may be isolated or obtained from the organism itself or medium used to culture or grow said organism.

As defined herein, the terms “whole broth culture” and “whole cell broth” refer to a liquid culture containing both cells and media. If bacteria are grown on a plate the cells can be harvested in water or other liquid, to provide a whole broth culture.

As defined herein, “filtrate” refers to liquid from a whole broth culture that has been passed through a membrane.

As defined herein, “extract” refers to liquid substance removed from cells by a solvent (water, detergent, buffer) and separated from the cells by centrifugation, filtration or other method.

As defined herein “excipient” as defined herein is an inert, organic or inorganic material, with which the active ingredient is mixed or formulated to facilitate its application to a plant or other object to be treated, or to facilitate its storage, transport and/or handling.

As used herein the term “effective amount” means an amount sufficient for beneficial effects or desired results. In terms of prevention and inhibition, an “effective amount” is the amount sufficient to palliate, ameliorate, stabilize, slow or delay the progression of fungal disease.

In an embodiment of the present invention rhizospheric and endophytic bacteria are isolated from many locations through selective plating techniques. The soil samples are subjected to serial dilution and plated on different media such as Nutrient agar, Kings B agar, Hi Chrome Bacillus agar and various other medium. Different cultures are shortlisted based on colony morphology, microscopy, biochemical and molecular characterization, and in vitro evaluation studies. These cultures are characterized at molecular level and its 16s rRNA are sequenced. The details of the shortlisted culture of various novel strains of Bacillus spp. is provided below in Table 1.

TABLE 1
Isolated strains of Bacillus species with antagonistic activity

| S. No. | MTCC code | Identity of the isolate |
|---|---|---|
| 1 | MTCC 25554 | Bacillus amyloliquefaciens |
| 2 | MTCC 25552 | Bacillus subtilis subsp. |
| | | Spizizenii |
| 3 | MTCC 25555 | Bacillus amyloliquefaciens |
| 4 | MTCC 25553 | Bacillus velezensis |
| 5 | MTCC 25556 | Bacillus safensis subsp. |
| | | Safensis |

Whole Genome Sequence of B. safensis—MTCC 25556:

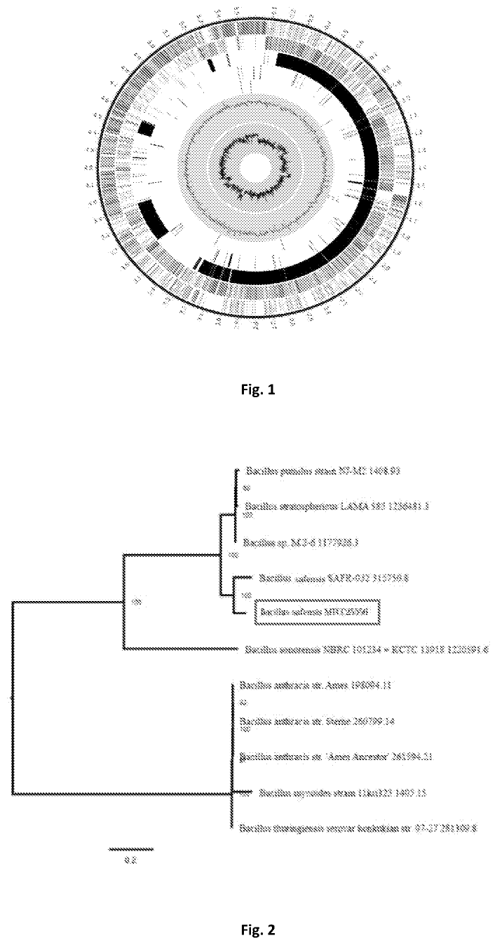

Genome assembly of B. safensis—MTCC 25556 was done by using SPAdes (Bankevich et al., 2012). This assembled genome had 1 contig, with the total length of 5,597,990 bp (5.5 Mb) and an average G+C content of 41.76% (FIG. 1). The closest reference and representative genomes were identified by Mash/MinHash. PATRIC global protein families (PGFams) were selected from these genomes to determine the phylogenetic placement of this genome (FIG. 2). RAST tool kit (RASTtk) was used to annotate Bacillus safensis MTCC 25556. This genome has 5,674 protein coding sequences (CDS), 152 transfer RNA (tRNA) genes, and 45 ribosomal RNA (rRNA) genes. The annotation included 1,191 hypothetical proteins and 4,483 proteins with functional assignments. The proteins with functional assignments included 1,347 proteins with Enzyme Commission (EC) numbers, 1,110 with Gene Ontology (GO) assignments, and 983 proteins that were mapped to KEGG pathways. There are 81 subsystems and 850 genes involved in metabolism. Protein processing involves 43 subsystems and 336 genes. In stress response, defence, and virulence, there are 27 subsystems and 194 genes implicated. 27 subsystems and 283 genes are engaged in cellular processes. There are 18 subsystems and 115 genes involved in DNA processing. There are 13 subsystems and 78 genes involved in RNA processing. Furthermore, 15 subsystems and 115 genes are involved in membrane transport.

This genome has 17 regions encoding secondary metabolite clusters like siderophore, terpene, RRE-containing, NRPS, T1PKS, RiPP-like, beta lactone, T3PKS, thiopeptide, LAP and other. Additionally, it contains 18 CDS encoding flagellant (Flg), 2 CDS encoding Elongation factor tubulin (Ef-Tu), 9 CDS encoding Peptidoglycan (PGN), 3 CDS encoding glucanase related genes, 3 CDS encoding thiol peroxidase (Tpx), and 5 CDS encoding Phosphate ABC transporter binding protein. Furthermore, 6 SSRs were identified; SSR 1 consists of (T)10, starts from 227820 to 227829; SSR2 contains (T)10 with spanning of 266094 to 266103; SSR3 consists of (A)10, from 569867 to 569876; SSR4 contains (T)10 and spans therange of 638362 to 638371; SSR5 includes (A)16 from 816043 to 816058; SSR6 contains (T)10 with a range of 1835177 to 1835186.

Whole Genome Sequence of B. amyloliquefaciens—MTCC 25554:

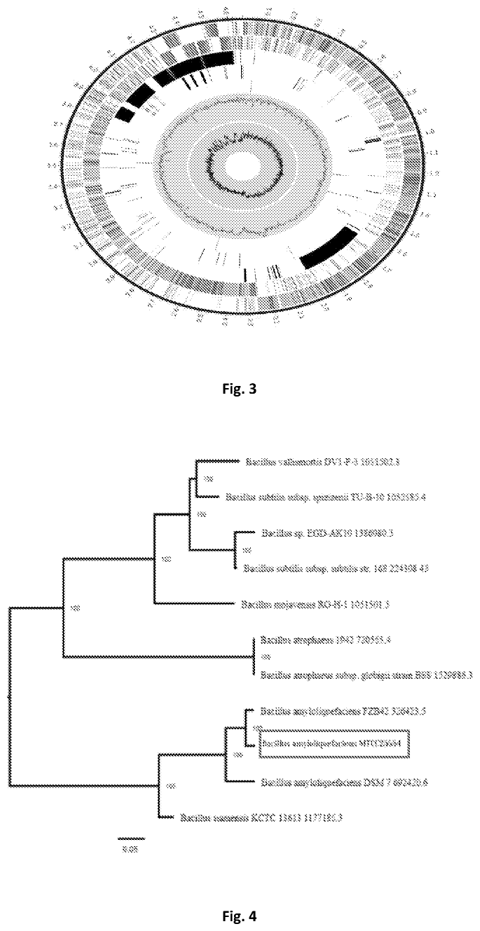

Genome assembly of B. amyloliquefaciens—MTCC 25554 was done by using SPAdes (Bankevich et al., 2012). This assembled genome had 1 contig, with the total length of 4,662,442 bp (4.6 Mb) and an average G+C content of 46.68% (FIG. 3). The closest reference and representative genomes to were identified by Mash/MinHash. PATRIC global protein families (PGFams) were selected from these genomes to determine the phylogenetic placement of this genome (FIG. 4). RAST tool kit (RASTtk) was used to annotate Bacillus amyloliquefaciens—MTCC 25554. This genome has 4,751 protein coding sequences (CDS), 168 transfer RNA (tRNA) genes, and 35 ribosomal RNA (rRNA) genes. The annotation included 818 hypothetical proteins and 3,933 proteins with functional assignments. The proteins with functional assignments included 1221 proteins with EC numbers, 1022 with GO assignments, and 911 proteins that were mapped to KEGG pathways. There are 93 subsystems and 901 genes involved in metabolism. Protein processing involves 41 subsystems and 374 genes. In stress response, defence, and virulence, there are 34 subsystems and 187 genes implicated. 29 subsystems and 318 genes are engaged in cellular processes. There are 16 subsystems and 86 genes involved in DNA processing. There are 13 subsystems and 61 genes involved in RNA processing. Furthermore, 16 subsystems and 73 genes are involved in membrane transport.

This genome has 14 regions encoding secondary metabolite clusters like TransAT-PKS, terpene, NRPS, T3PKS, RiPP-like, betalactone, lanthipeptide-class-ii, PKS-like and other. Additionally, it contains 44 CDS encoding flagellin (Flg), 3 CDS encoding Elongation factor tubulin (Ef-Tu), 8 CDS encoding Peptidoglycan (PGN), 2 CDS encoding glucanase related genes, 2 CDS encoding xylanase related genes, 12 CDS encoding arabinase related genes, 2 CDS encoding amylase related genes, 6 CDS encoding glucosidase related genes, 1 CDS for chitin binding protein related genes, 1 CDS for chitosanase related gene, 2 CDS encoding thiol peroxidase (Tpx), 4 CDS encoding Phosphate ABC transporter binding protein, 15 CDS encoding oligopeptide ABC transporter related genes, 3 CDS for acetolactate synthase related genes, 1 CDS encoding acetolactate decarboxylase related genes and 5 acetoin dehydrogenase related genes. Furthermore, 5 SSRs were identified; SSR 1 consists of (A)10, starts from 757378 to 757387; SSR2 contains (T)10 with spanning of 859776 to 859785; SSR3 consists of (T)10, from 1266960 to 1266969; SSR4 contains (T)10 and spans the range of 1305041 to 1305050; SSR5 includes (AAG)5 from 1337573 to 1337587.

Whole Genome Sequence of Bacillus subtilis Subsp. Spizizenii—MTCC 25552

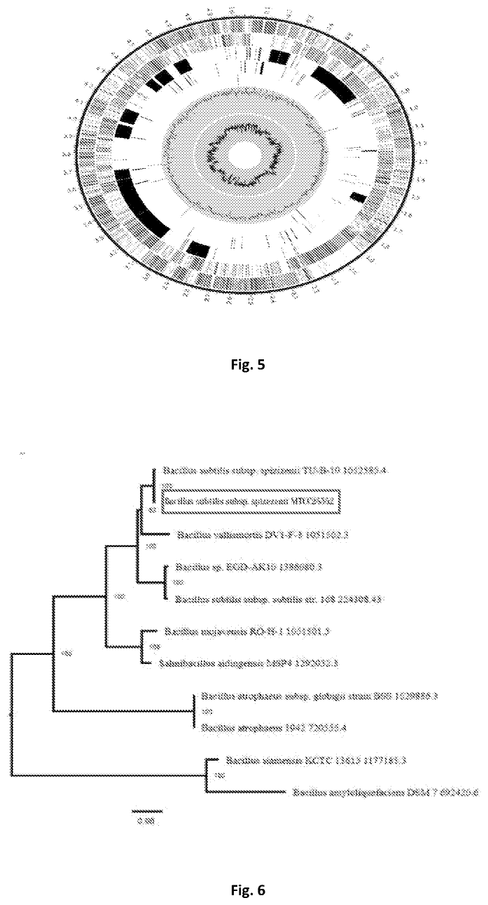

Genome assembly of B. subtilis subsp. Spizizenii—MTCC 25552 was done by using SPAdes (Bankevich et al., 2012). This assembled genome had 1 contig, with the total length of 5,057,534 bp (5.0 Mb) and an average G+C content of 44.01% (FIG. 5). The closest reference and representative genomes to were identified by Mash/MinHash. PATRIC global protein families (PGFams) were selected from these genomes to determine the phylogenetic placement of this genome (FIG. 6). RAST tool kit (RASTtk) was used to annotate B. subtilis subsp. spizizenii—MTCC 25552. This genome has 5,204 protein coding sequences (CDS), 106 transfer RNA (tRNA) genes, and 30 ribosomal RNA (rRNA) genes. The annotation included 793 hypothetical proteins and 4,411 proteins with functional assignments. The proteins with functional assignments included 1298 proteins with EC numbers, 1077 with GO assignments, and 961 proteins that were mapped to KEGG pathways. There are 98 subsystems and 952 genes involved in metabolism. Protein processing involves 43 subsystems and 274 genes. In stress response, defence, and virulence, there are 34 subsystems and 196 genes implicated. 29 subsystems and 299 genes are engaged in cellular processes. There are 18 subsystems and 108 genes involved in DNA processing. There are 13 subsystems and 66 genes involved in RNA processing. Furthermore, 15 subsystems and 88 genes are involved in membrane transport.

This genome has 14 regions encoding secondary metabolite clusters like TransAT-PKS, terpene, NRPS, T3PKS, betalactone, lanthipeptide-class-i, Sactipeptide, CDPS, PKS-like and other. Additionally, it contains 3 CDS encoding flagellin (Flg), 1 CDS encoding Elongation factor tubulin (Ef-Tu), 3 CDS encoding Peptidoglycan (PGN), 2 CDS encoding glucanase related genes, 2 CDS encoding xylanase related genes, 2 CDS encoding arabinase related genes, 1 CDS encoding amylase related genes, 1 CDS encoding glucosidase related genes, 1 CDS for chitin binding protein related genes, 1 CDS for chitosanase related gene, 3 CDS encoding thiol peroxidase (Tpx), 5 CDS encoding Phosphate ABC transporter binding protein, 12 CDS encoding oligopeptide ABC transporter related genes, 6 CDS for acetolactate synthase related genes, 2 CDS encoding acetolactate decarboxylase related genes and 4 acetoin dehrdrogenase related genes. Furthermore, 8 SSRs were identified; SSR 1 consists of (C)13, starts from 878845 to 878857; SSR2 contains (GCC)5 with spanning of 1066887 to 1066901; SSR3 consists of (GCC)5, from 1067037 to 1067051; SSR4 contains (T)10 and spans the range of 1078183 to 1078192; SSR5 includes (GTT)5 from 1334938 to 1334952. SSR6 includes (A)10 from 1343833 to 1343842. SSR7 includes (T)10 from 1606636 to 1606645. SSR8 includes (T)10 from 1634189 to 1634198.

Whole Genome Sequence of Bacillus amyloliquefaciens—MTCC 25555:

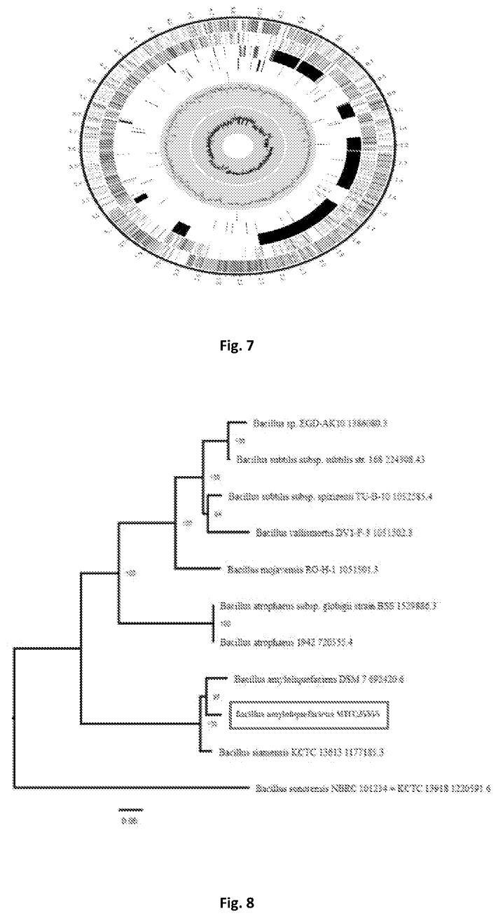

Genome assembly of B. amyloliquefaciens—MTCC 25555 was done by using SPAdes (Bankevich et al., 2012). This assembled genome had 1 contig, with the total length of 7,703,262 bp (7.7 Mb) and an average G+C content of 46.50% (FIG. 7). The closest reference and representative genomes to were identified by Mash/MinHash. PATRIC global protein families (PGFams) were selected from these genomes to determine the phylogenetic placement of this genome (FIG. 8). RAST tool kit (RASTtk) was used to annotate B. amyloliquefaciens—MTCC 25555. This genome has 7,726 protein coding sequences (CDS), 169 transfer RNA (tRNA) genes, and 43 ribosomal RNA (rRNA) genes. The annotation included 1323 hypothetical proteins and 6,403 proteins with functional assignments. The proteins with functional assignments included 1972 proteins with EC numbers, 1645 with GO assignments, and 1463 proteins that were mapped to KEGG pathways. There are 93 subsystems and 1400 genes involved in metabolism. Protein processing involves 43 subsystems and 426 genes. In stress response, defence, and virulence, there are 34 subsystems and 286 genes implicated. 29 subsystems and 480 genes are engaged in cellular processes. There are 17 subsystems and 157 genes involved in DNA processing. There are 13 subsystems and 102 genes involved in RNA processing. Furthermore, 16 subsystems and 138 genes are involved in membrane transport.

This genome has 26 regions encoding secondary metabolite clusters like TransAT-PKS, terpene, TransAT-PKS-like, NRPS, RiPP-like, T3PKS, betalactone, RRE-Containing, LAP, PKS-like and other. Additionally, it contains 4 CDS encoding flagellin (Flg), 2 CDS encoding Elongation factor tubulin (Ef-Tu), 8 CDS encoding Peptidoglycan (PGN), 4 CDS encoding glucanase related genes, 4 CDS encoding xylanase related genes, 4 CDS encoding arabinase related genes, 2 CDS encoding amylase related genes, 2 CDS encoding glucosidase related genes, 2 CDS for chitin binding protein related genes, 2 CDS for chitosanase related gene, 4 CDS encoding thiol peroxidase (Tpx), 10 CDS encoding Phosphate ABC transporter binding protein, 24 CDS encoding oligopeptide ABC transporter related genes, 6 CDS for acetolactate synthase related genes, 2 CDS encoding acetolactate decarboxylase related genes and 8 acetoin dehydrogenase related genes. Furthermore, 6 SSRs were identified; SSR 1 consists of (CAT)5, starts from 1312630 to 1312644; SSR2 contains (T)10 with spanning of 1362805 to 1362814; SSR3 consists of (CAT)5, from 1578235 to 1578249; SSR4 contains (T)10 and spans the range of 1628410 to 1628419; SSR5 includes (A)14 from 1751722 to 1751735. SSR6 includes (GCT) 5 from 1791842 to 1791856.

### Whole Genome Sequence of Bacillus Velezensis—MTCC 25553:

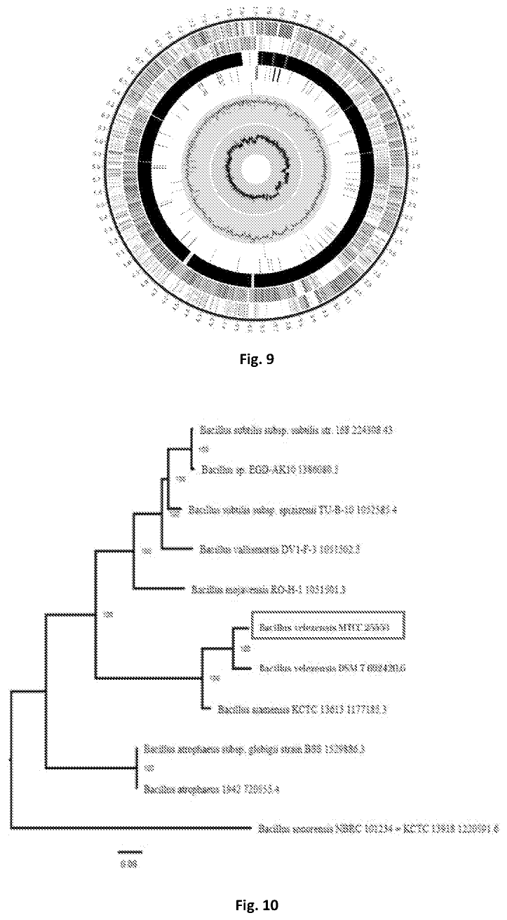

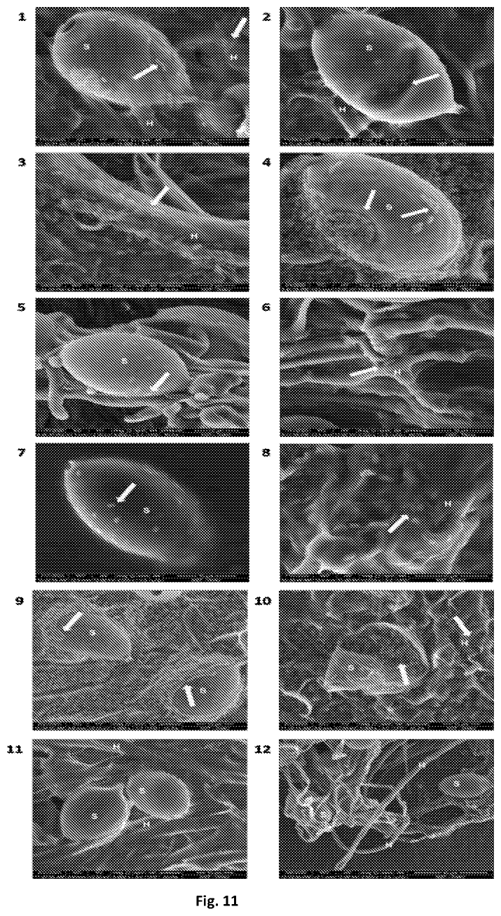

Genome assembly of B. velezensis—MTCC 25553 was done by using SPAdes (Bankevich et al., 2012). This assembled genome had 1 contig, with the total length of 4,820,496 bp (4.8 Mb) and an average G+C content of 46.46% (FIG. 9). The closest reference and representative genomes to were identified by Mash/MinHash. PATRIC global protein families (PGFams) were selected from these genomes to determine the phylogenetic placement of this genome (FIG. 10). RAST tool kit (RASTtk) was used to annotate B. velezensis—MTCC 25553. This genome has 4,814 protein coding sequences (CDS), 131 transfer RNA (tRNA) genes, and 66 ribosomal RNA (rRNA) genes. The annotation included 867 hypothetical proteins and 3,947 proteins with functional assignments. The proteins with functional assignments included 1200 proteins with EC numbers, 994 with GO assignments, and 878 proteins that were mapped to KEGG pathways. There are 93 subsystems and 861 genes involved in metabolism. Protein processing involves 43 subsystems and 286 genes. In stress response, defence, and virulence, there are 34 subsystems and 205 genes implicated. 29 subsystems and 261 genes are engaged in cellular processes. There are 17 subsystems and 87 genes involved in DNA processing. There are 13 subsystems and 68 genes involved in RNA processing. Furthermore, 16 subsystems and 87 genes are involved in membrane transport.

This genome has 17 regions encoding secondary metabolite clusters like TransAT-PKS, terpene, NRPS, RiPP-like T3PKS, LAP, RRE-containing, betalactone, PKS-like and other. Additionally, it contains 2 CDS encoding flagellin (Flg), 2 CDS encoding Elongation factor tubulin (Ef-Tu), 3 CDS encoding Peptidoglycan (PGN), 2 CDS encoding glucanase related genes, 2 CDS encoding xylanase related genes, 2 CDS encoding amylase related genes, 2 CDS encoding glucosidase related genes, 1 CDS for chitin binding protein related genes, 1 CDS for chitosanase related gene, 2 CDS encoding thiol peroxidase (Tpx), 5 CDS encoding Phosphate ABC transporter binding protein, 14 CDS encoding oligopeptide ABC transporter related genes, 3 CDS for acetolactate synthase related genes, 1 CDS encoding acetolactate decarboxylase related genes and 3 acetoin dehyrdrogenase related genes. Furthermore, 4 SSRs were identified; SSR 1 consists of (A)10, starts from 1084388 to 1084397; SSR2 contains (GAT)5 with spanning of 1134557 to 1134571; SSR3 consists of (GCT)5, from 1290637 to 1290651; SSR4 contains (GCT)5 and spans the range of 1439945 to 1439959.

The antagonistic bacteria are not limited to but may include species of the following genera of microorganisms: Alcanivorax, Aromatoleum, Bifidobacterium, Dechloromonas, Lactobacillus, Acetobacter, Acinetobacter, Aerobacter, Agrobacterium, Arthrobacter, Azospirillum, Azotobacter, Bacillus, Bifidobacterium, Burkholderia, Dehalococcoides, Desulfitobacterium, Frankia, Frateuria, Geobacter, Geothrix, Klebsiella, Lactobacillus, Lysobacter, Methanobacillus, Methanosarcina, Methylobacterium micromonospora, Ochrobactrum, Pantoea, Pseudomonas, Rhizobium, Shewanella, Sinorhizobium, Streptococcus, Streptomyces, Thiobacillus, Zymomonas, etc.

In another embodiment of the present invention, the isolated strains of Bacillus spp. are screened for antagonistic activity against Phytophthora infestans through a dual culture technique. The uniqueness about the isolated cultures of the present invention is that the bacterial cultures are able to grow and control the pathogen at 19° C. which is not an optimum growth condition for the bacteria. The isolated strains of Bacillus spp. are found to exhibit 95% to 100% inhibition (antagonistic activity) against Phytophthora infestans.

The isolated strains of Bacillus spp., show their antagonistic activity through antimicrobial peptides. Antimicrobial peptides such as Subtilosin (alb A), Mycosubtulin (myc C), Bacilysin (BacAB, CD), Ericin (Eri B), Bacillomycin (Bam C), Fengycin (fen B), Subtilosin (alb F), Fengycin (fen CAE), Iturin (ipa 14), Iturin C (Itu C), Mersacidin (mrs A), Iturin (ItuD, D1), Subtilin(SPCA, S), Surfactin (srf AB) have been elucidated through molecular characterization of the bacterial isolates.

In another embodiment of the present invention, the microbial formulation may comprise either a single strain of isolated Bacillus spp., or a binary, ternary mixture of isolated Bacillus spp. as illustrated in Table 1 along with excipients.

In yet another embodiment, the present invention provides method of preparing formulation with the isolated effective bacterial isolates listed in Table 1 against Phytophthora infestans that comprises a critical step of concentration of active ingredient. Concentration of active ingredient plays a critical factor in determining the stability of the formulation. Cell count of the culture is increased using “Tangential Flow Filtration” (TFF). TFF technique involves the passing of the fermented broth through micro fibre filters. This process helps in achieving a concentration of up to 10 times the original volume of the fermented broth. The Colony Forming Unit/ml increased from 1×10⁸-1×10⁹ to 1×10⁹-1×10¹⁰ as shown in Table 2.

TABLE 2
Development of high cell count
concentrate of antagonistic bacterial culture
Cell Count (Colony Forming Units- CFU/ml)

| CFU before TFF | CFU after TFF |
|---|---|
| 1 × 10⁸-1 × 10⁹ | 1 × 10⁹-1 × 10¹⁰ |

Upon completion of the Tangential-flow filtration, the formulation preparation consisted of the active ingredient which is the concentrated cell mass of bacteria not limited to but ranging from 1 to 98% by wt.

The formulation may also contain surfactants such as Polyethylene glycol (PEG)-100, PEG-200, PEG-300, PEG-400, PEG-8000, PEG-20000, Tween-20, Tween-40, Tween-60, Tween-80, Span-20, Span-40, PEG 6000, glycerol, PVP K30, Tween 85 and others. The range of the surfactants may range from 0.001 to 31% (wt./wt.) of the formulation.

The formulation may also contain encapsulant. The encapsulant is selected from the group comprising glycerol, PEG (Polyethylene glycol) and PVP (Polyvinylpyrrolidone), preferably, glycerol present in a range from 2 to 5% (wt./wt.).

The formulation may also contain sticking agents not limited to xanthun gum, 0.4% xanthan gum, Guar gum, Gum acacia, Arabic gum, Malto dextrin, starch soluble and insoluble, Carboxy methyl cellulose and others. The quantity of the sticking agents may range from 0.05 to 82% (wt./wt.) of the formulation, preferably, the sticking agent is xanthan gum.

The formulation also consists of adjuvants not limited to oils such as Olive oil, Cashew oil, Castor oil, Sunflower oil, Pongamia oil, Sesame oil, Linseed oil, Rice Bran oil, Ground nut oil and other oils of plant and animal origin. The quantity of adjuvants ranges from 2 to 98% (wt./wt.) of the formulation, preferably, the adjuvant is linseed oil and sesame oil.

The formulation may also contain preservatives such as citric acid, calcium propionate, copper sulphate, magnesium sulphate, boric acid, calcium benzoate, molybdenum or combination thereof present in a range from 0.01 to 0.25% (wt./wt.).

In yet another embodiment, formulations prepared with a single strain of Bacillus spp. alone or a combination of strains and subjected to real time shelf-life studies on a monthly basis and were found to be stable during shelf life of eight months.

The microbial formulations can be formulated in any manner. Exemplary formulations include but are not limited to aqueous suspension (AS), emulsifiable concentrates (EC), wettable powders (WP), soluble liquids (SL), aerosols, ultra-low volume concentrate solutions (ULV), soluble powders (SP), microencapsulates, water-dispersed granules, flowables (FL), micro emulsions (ME), Nano-emulsions (NE), etc. The preferred formulation is aqueous suspension (AS) or emulsifiable concentrates (EC) in which the formulations of the present invention are prepared. In any formulation of the present invention, percent of the active ingredient is within a range of 0.5% to 98% by wt. of the formulation.

In an embodiment, the present invention provides a method of preventing or inhibiting fungus infections on plants comprising applying an effective amount of the novel strain or the mutant.

The following examples are provided to illustrate the invention. These examples are not to be construed as limiting.

### Example 1: Documentation of Ultra-Microscopic Changes of P. infestans Morphology Under Scanning Electron Microscope (SEM): Di-Trophic Interaction

a) Marginal mycelium of P. infestans interacted with bacterial endophytes and healthy mycelium from control plate, were subjected for SEM documentation. Sporangia and hyphal morphologies were mainly focused to identify the changes in P. infestans mycelium interacted with bacterial endophytes and healthy mycelium.
b) All the five effective bacterial endophytes were able to colonize and hyperparasites the sporangia and hyphal structures of P. infestans (FIG. 11). B. safensis—MTCC 25556, B. amyloliquefaciens—MTCC 25554, and B. amyloliquefaciens—MTCC 25555 showed the severe morphological alterations on P. infestans. The sporangia were shrunken and sporangial wall was destructed by bacterial colonization. In hypha, loss of cell wall integration, protrusions and less hyphal content were observed. In control, clear sporangia and hypha were observed.

### Example 2: Colonization Potential of Bacterial Endophytes

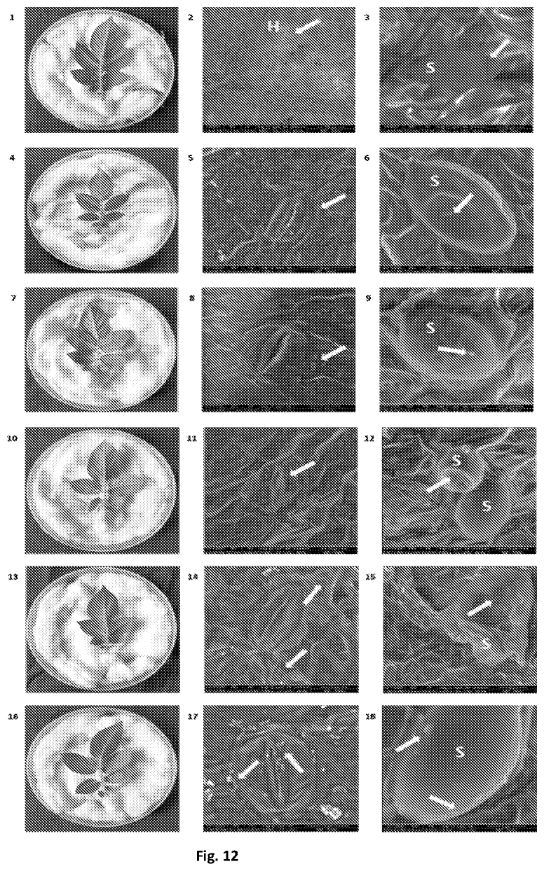

Detached leaf assay was performed to determine the colonization potential and antagonistic activity of bacterial endophytes against P. infestans. Forty days old leaves were collected from potato cv. Kufri Jyothi and placed on sterilized cotton in petri dish (200×20 mm). Inoculated bacterial cell suspension (10⁸ CFU/ml) to the leaves first and then sporangial suspension of P. infestans (10⁶ sporangia/ml) was added, whereas in inoculated control, only sporangial suspension was sprayed as a positive control. After inoculation, petri dishes were kept in dark conditions at 19±2° C. for 7 days. The observations were documented using SEM (FIG. 12). In inoculated control, the pathogen, P. infestans successfully established connection with the host i.e., potato leaf and symptoms were expressed, whereas in bacterial endophytes along with P. infestans treated leaves, establishment of P. infestans was not seen, instead bacterial colonization on leaf and pathogen propagules were observed, which confirms the colonization potential on potato leaf and antagonistic activity of bacterial endophytes.

### Example 3: Bio-Fungicidal Formulations

The different components and their weight % ranges in the bio-fungicides formulation are illustrated in Table 3. The specific formulations but not limited thereto for the purpose of illustration of the present invention are shown in Table 4.

Table 4 is sample representation of one culture Bacillus amyloliquefaciens—MTCC 25554.

TABLE 3
Components in bio-fungicides

| S. | Component | | |
|---|---|---|---|
| No | type | Particular | Range (%) |
| 1 | Active | Bacillus amyloliquefaciens -MTCC | 1-98 |
| | ingredient | 25554/, | |
| | | Bacillus subtilis subsp. spizizenii - | |
| | | MTCC 25552/, | |
| | | Bacillus amyloliquefaciens - MTCC | |
| | | 25555/, | |
| | | Bacillus velezensis - MTCC 25553/, | |
| | | Bacillus safensis subsp. safensis - | |
| | | MTCC 25556. | |
| 2 | Surfactant | PEG 6000 | 0.5-2 |
| 3 | Surfactant/ | Glycerol | 2-5 |
| | Encapsulant | | |
| 4 | Surfactant | PVP K30 | 0.1-5 |
| 5 | Surfactant | PEG 400 | 2-20 |
| 6 | Adjuvant | Linseed oil | 5-98 |
| 7 | Adjuvant | Sesame oil | 2-52 |
| 8 | Surfactant | Tween 85 | 1-31 |
| 9 | Surfactant | Tween 80 | 0.001-0.01 |
| 10 | Sticking | Xanthan gum | 0.05-0.33 |
| | agents | | |
| 11 | Sticking | 0.4% Xanthan gum | 0-82 |
| | agents | | |
| 12 | Preservative | Citric acid | 0.1-0.25 |
| 13 | Preservative | Calcium propionate | 0.1-0.2 |
| 14 | Preservative | Copper sulphate | 0.01-0.05 |
| 15 | Water | Water | Q.S. |

TABLE 4
Formulations for the bio-fungicide formulation

| | Formulations (% wt./wt.) | | | | | |
|---|---|---|---|---|---|---|
| | 1 | 2 | 3 | 4 | 5 | 6 |
| Active ingredient: | 1 | 10 | 3 | 98 | 1 | 50 |
| Bacillus amyloliquefaciens - | | | | | | |
| MTCC 25554/, | | | | | | |
| Bacillus subtilis subsp. | | | | | | |
| spizizenii - MTCC 25552/, | | | | | | |
| Bacillus amyloliquefaciens - | | | | | | |
| MTCC 25555/, | | | | | | |
| Bacillus velezensis - MTCC | | | | | | |
| 25553/, Bacillus safensis | | | | | | |
| subsp. safensis - MTCC | | | | | | |
| 25556. | | | | | | |
| PEG 600 | 0.5 | 2 | 1 | 0 | 0 | 0 |
| Glycerol | 2.5 | 5 | 4 | 2 | 0 | 0 |
| PVP K30 | 0.1 | 0.5 | 5 | 0 | 0 | 1 |
| PEG 400 | 20 | 0 | 2 | 0 | 0 | 0 |
| Linseed oil | 5 | 0 | 52 | 0 | 98 | 0 |
| Sesame oil | 52 | 0 | 2 | 0 | 0 | 0 |
| Tween 85 | 18.9 | 0 | 31 | 0 | 1 | 0 |
| Tween 80 | 0 | 0 | 0 | 0 | 0 | 0.01 |
| 0.4% Xanthan gum | 0 | 82 | 0 | 0 | 0 | 0 |
| Xanthan gum | 0 | 0 | 0 | 0 | 0 | 0.05 |
| Citric acid | 0 | 0.25 | 0 | 0 | 0 | 0 |
| Calcium Propionate | 0 | 0.2 | 0 | 0 | 0 | 0 |
| Copper sulphate | 0 | 0.05 | 0 | 0 | 0 | 0 |
| Water | q.s. | q.s. | q.s. | q.s. | q.s. | 48.94 |

### Example 4: Preparation of Bacterial Formulation—Aqueous Formulation

The example is a representative illustration for preparing an aqueous formulation of 300 ml. The preparation comprises of following steps:
a) 500 ml clean beaker is taken and required amount of sticking agent as per formulation is weighed in accordance with Table 4 & 300 ml of aqueous carrier is added and mixed well with a stirrer at ambient temperature and atmospheric pressure.
b) The beaker is kept in boiling (1000±1° C.) water bath and mixed well to dissolve the sticking agent.
c) After that the dissolved sticking agent solution from step b) is poured into 500 ml screw cap bottle and kept for sterilization. Sterilization is carried out at 121° C. for 20 minutes at 15 psi of pressure.
d) The sterilized sticking agent solution is cooled up to room temperature.
   The following steps (steps: e-j) are performed under sterile conditions. The steps (steps: e-j) are carried inside Laminar Air Flow chamber to maintain sterile conditions.
e) A sterile 500 ml beaker is placed on weighing balance in a sterile air chamber & tared.
f) Required quantity of active ingredient is weighed and required quantity of encapsulant is added as per formulation illustrated in Table 4 (for encapsulation of active ingredient) using sterile spatula.
g) Required quantity of preservative is added as per formulation illustrated in Table 4 using sterile pipette & mixed well using sterile glass rod until paste formation.
h) After that required quantity of sticker is added & volume is made up to 300 g using sterile sticking agent.
i) Preservatives are added as per formulation and mixed with the formulation with high-speed stirrer at 6000 RPM for 10 min.
j) After mixing, the formulation is equally distributed into sterile pet bottles (15 Nos.) approximately 20 ml each for final field use.

For industrial scale preparation, autoclavable formulation vessels with agitation and temperature control is being used. Either the formulation ingredients are placed in formulation vessels and sterilized, or the pre-sterilized ingredients are added to the formulation vessels, mixed with active ingredient and filled under sterile conditions in suitable containers.

### Example 5: Preparation of Bacterial Formulation—Oil Formulation (Emulsifiable Concentrate) (300 ml)

The process comprises of following steps:
a) 500 ml clean screw cap bottle is taken and required oil carrier & surfactant are separately weighed as per formulation in Table 4.
b) The vegetable oils and surfactants are sterilized at 1000±1° C. for 30 min using Autoclave.
c) After sterilization the vegetable oils and surfactants are cooled up to room temperature.

The following steps (steps: d-i) are performed under sterile conditions.
d) The sterile 500 ml beaker is taken and put on weighing balance in a sterile air chamber/LAF (Laminar Air Flow chamber) & tare.
e) Required quantity of active ingredient is taken and required quantity of encapsulant is added as per formulation illustrated in Table 4 (for encapsulation of active ingredient) using sterile spatula.
f) Required quantity of preservative is added as per formulation in Table 4 using sterile pipette & mixed well using sterile glass rod until paste formation.
g) Required quantity of sterile oil carrier, surfactant & preservative is added as per formulation shown in Table 4.
h) The formulation is mixed with high-speed stirrer at 6000 RPM for 10 min.
i) After mixing the formulation is equally distributed into sterile pet bottles (15 Nos.) approximately 20 ml each.

### Example 6: Efficacy of the Bio-Fungicide Formulation is the Present Invention

### Evaluation of Effective Bacterial Endophytes Against Late Blight of Potato Under Field Conditions:

The field experiments were conducted at Thummanatti under Nilgiris district during 2021-22 to study the field efficacy of bacterial endophytes against late blight disease of potato. The experimental site is situated at 11° 25′N latitude and 76° 45′E longitude under the southern hill regions of Tamil Nadu, India. The details of experiment were mentioned in Table 5.

TABLE 5
Evaluation of consortia bacterial endophytes against late blight of potato under field conditions:

| | | | Per cent | Per cent |
|---|---|---|---|---|
| | | | Disease | reduction |
| Tr. | | Dose | Index | over |
| No. | Treatments | mL/Litre | (PDI)% | control |
| T1 | Tuber dip with B. subtilis subsp. spizezinii - MTCC 25552 + | 20 + 10 | 17.8 | 81.1 |
| | B. velezensis - MTCC 25553 + B. amyloliquefaciens - | | | |
| | MTCC 25554 + B. amyloliquefaciens - MTCC 25555 + B. | | | |
| | safensis subsp. safensis - MTCC 25556 + Soil drenching | | | |
| | and foliar application (30, 45, 60 and 75th day) | | | |
| T2 | Tuber dip with B. subtilis subsp. spizezinii - MTCC 25552 + | 20 + 15 | 16.9 | 82 |
| | B. velezensis - MTCC 25553 + B. amyloliquefaciens - | | | |
| | MTCC 25554 + B. amyloliquefaciens - MTCC 25555 + B. | | | |
| | safensis subsp. safensis - MTCC 25556 + Soil drenching | | | |
| | and foliar application (30, 45, 60 and 75th day) | | | |
| T3 | Foliar spray of Mandipropamid for late blight of potato | 2.0 | 12.5 | 86.7 |
| | (30, 45, 60 and 75^{th} day) | | | |
| T4 | Foliar spray of Mancozeb for late blight of potato (30, 45, | 2 g/lit | 15.6 | 83.4 |
| | 60 and 75^{th} day) | | | |
| T5 | Foliar spray of Mancozeb (45^{th} day) | 2 g/lit | 45.2 | 51.9 |
| T6 | Farmer's practice | — | 78.4 | 16.6 |
| T7 | Untreated control | | 94 | 0 |
| | CD (p = 0.05) 2.49 | | | |

### Evaluation of Bacterial Endophytes Against Late Blight of Potato Under Field Conditions:

The field experiments were conducted at Ithalar under Nilgiris district during 2021-22 to study the field efficacy of bacterial endophytes against late blight disease of potato. The experimental site is situated at 11° 20′N latitude and 76° 36′E longitude under the southern hill regions of Tamil Nadu, India. The details of experiment were mentioned in Table 6.

The formulation 2 from Table 4 with consortia antagonistic bacteria was tested for control of Phytophthora infestans. Tuber dip with endophytic bacteria consortia formulation (B. subtilis subsp. spizezinii—MTCC 25552+B. velezensis—MTCC 25553+B. amyloliquefaciens—MTCC 25554+B. amyloliquefaciens—MTCC 25555+B. safensis subsp. safensis—MTCC 25556)+Soil drenching and foliar application @35 ml and 30 ml per litre on 30, 45, 60 and 75th day after planting had the least PDI of 16.9% and 17.8%, respectively. Both the doses were found statistically on par to each other in the suppression of late blight of potato under field conditions.

The disease control of consortia formulation was almost effective to chemical treatment of Mandipropamid and Mancozeb where the PDI was recorded as 12.5% and 15.6%, respectively as shown in Table 5. However, the maximum incidence of disease 94.0% was observed in untreated control.

The results as illustrated in Table 6 clearly shows that by foliar spray of Bacillus consortia (30 ml/liter @10¹⁰ cfu/ml) on 5 days interval at 30, 35, 40 and 45 days after planting was effective in the suppression of late blight of potato. Our bio fungicide formulation was superior and on par with commercial Pesticide products.

TABLE 6
Evaluation of bacterial endophytes against late
blight of potato under field conditions:

| | | | Per cent | |
|---|---|---|---|---|
| | | | Disease | Per cent |
| Tr | | | Index | reduction |
| No. | Treatments | Dose | (PDI) | over control |
| T1 | Untreated control | — | 85.00 | 0.00 |
| T2 | Tuber dip with B. subtilis | 30 ml/L | 16.9 | 80.11 |
| | subsp. spizezinii - MTCC | | | |
| | 25552 + B. velezensis - | | | |
| | MTCC 25553 + B. | | | |
| | amyloliquefaciens - MTCC | | | |
| | 25554 + B. amyloliquefaciens - | | | |
| | MTCC 25555 + B. safensis | | | |
| | subsp. Safensis - MTCC | | | |
| | 25556 + Soil drenching and | | | |
| | foliar application (5 days | | | |
| | interval) | | | |
| T3 | Tuber dip with B. subtilis | 30 ml/L | 18.9 | 77.76 |
| | subsp. spizezinii - MTCC | | | |
| | 25552 + B. velezensis - | | | |
| | MTCC 25553 + B. | | | |
| | amyloliquefaciens - MTCC | | | |
| | 25554 + B. amyloliquefaciens - | | | |
| | MTCC 25555 + B. safensis | | | |
| | subsp. Safensis - MTCC | | | |
| | 25556 + Soil drenching and | | | |
| | foliar application (7 days | | | |
| | interval) | | | |

| | |
|---|---|
| CD (p = 0.05) | 2.95 |

PDI- Values are originals from mean of three replications
Figures in the parentheses are arcsine transformed values

Commercial Pesticide products:

| | | | Per cent | Per cent |
|---|---|---|---|---|
| | | | Disease | reduction |
| Tr. | | | Index | over |
| No. | Treatments | Dose | (PDI) | control |
| T4 | Foliar spray of | 0.8 | 14.11 | 83.40 |
| | Mandipropamid (7 days | ml/L | | |
| | interval) | | | |
| T5 | Foliar spray of | 2 | 15.11 | 82.22 |
| | Ametoctradin | ml/L | | |
| | 27% + Dimethomorph | | | |
| | 20.27% w/w SC (7 days | | | |
| | interval) | | | |
| T6 | Mancozeb 75% WP (5 days | 4 | 19.76 | 76.75 |
| | interval) | g/L | | |

| | |
|---|---|
| CD (p = 0.05) | 2.95 |

PDI- Values are originals from mean of three replications
Figures in the parentheses are arcsine transformed values

### Example 7: Comparative Evaluation on the Efficacy of Bacillus Spp. Formulations of the Present Invention, with Commercial Products of Different Bacillus Spp. Against P. infestans Causing Late Blight of Potato

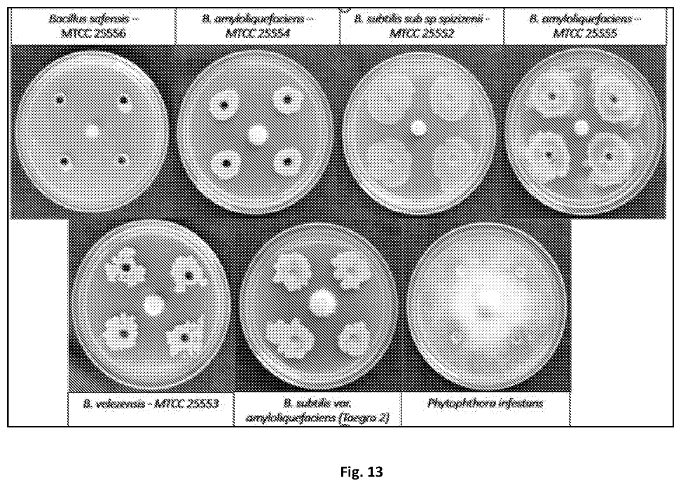

The best performing endophytes of Bacillus spp., in the present investigation was compared for their efficacy on the suppression of Phytophthora infestans with the international bioproducts including B. subtilis var. amyloliquefaciens (Taegro 2). Among the different bacterial antagonists compared, B. amyloliquefaciens—MTCC 25554 and B. safensis—MTCC 25556 inhibited the mycelial growth of P. infestans up to 99.33% over untreated control. The Percent inhibition of P. infestans mycelial growth (%) of these two cultures were superior when compared with global commercial products used in the evaluation as shown in Table 8 and FIG. 13.

TABLE 7
Details of commercial microbial products

| S. | | | | |
|---|---|---|---|---|
| No. | Product | Active Ingredient | Manufactured by | Distributed by |
| 1 | Taegro2 | Bacillus subtilis var. | Novozymes | Novozymes |
| | | amyloliquefaciens | Biologicals INC., | Biologicals |
| | | | USA | INC., USA |

TABLE 8
Comparative evaluation on the efficacy of Bacillus
spp. formulation of the present invention, with commercial
microbial products of different Bacillus spp. against
P. infestans causing Late blight of potato

| | | Product of | |
|---|---|---|---|
| | | present | Percent inhibition |
| | | invention/ | of P. infestans |
| | | Commercial | mycelial growth |
| S. No. | Isolate | product* | (%) |
| 1 | Bacillus safensis - MTCC | Present | 99.33^{a} |
| | 25556 | invention | (9.99) |
| 2 | B. amyloliquefaciens - MTCC | Present | 99.33^{a} |
| | 25554 | invention | (9.99) |
| 3 | B. velezensis - MTCC 25553 | Present | 98.00 ^{a} |
| | | invention | (9.92) |
| 4 | B. amyloliquefaciens - MTCC | Present | 98.00 ^{a} |
| | 25555 | invention | (9.92) |
| 5 | B. subtilis sub sp spizizenii - | Present | 95.66 ^{b} |
| | MTCC 25552 | invention | (9.80) |
| 6 | B. subtilis var. | Commercial | 82.00^{c} |
| | amyloliquefaciens (Taegro 2) | product | (9.08) |
| 7 | P. infestans (control) | | 0.00^{d} |
| | | | (0.71) |
| | CD@ 5% | | 0.084 |

Values in the parenthesis are square root of mean values
*Pure cultures isolated from commercial products

### Example 8: Experimental Data for Shelf Life of the Bio Fungicides Formulations

Formulations from Table 4 were subjected to real time shelf-life studies on a monthly basis. The formulations were stored at 28° C. and at a relative humidity of 60%. Aqueous based formulations are Formulation 1, 3, 5 and oil-based formulation are 2, 4, and 6. The CFU of the formulations were measured on a monthly basis to assess the viability of the cells throughout its storage period. It was found that aqueous formulation is stable with a minimum CFU of 1×10⁷/ml for a period of 10 months and oil formulation was stable for 7 months with a minimum CFU of 1×10⁷/ml as shown in Table 9.

TABLE 9
Stability of the bio fungicides formulations

| Formulation | CFU/ml | | | | | | | | | |
|---|---|---|---|---|---|---|---|---|---|---|
| No. | Month 1 | Month 2 | Month 3 | Month 4 | Month 5 | Month 6 | Month 7 | Month 8 | Month 9 | Month 10 |
| 1 | 5 × 10¹⁰ | 3 × 10¹⁰ | 1 × 10¹⁰ | 7 × 10⁹ | 9 × 10⁹ | 8 × 10⁸ | 5 × 10⁸ | 5 × 10⁷ | 6 × 10⁷ | 5 × 10⁷ |
| 2 | 4 × 10¹⁰ | 2 × 10¹⁰ | 1 × 10⁹ | 3 × 10⁹ | 7 × 10⁸ | 6 × 10⁸ | 1 × 10⁸ | 4 × 10⁷ | 4 × 10⁷ | 1 × 10⁷ |
| 3 | 1 × 10¹⁰ | 1 × 10¹⁰ | 8 × 10⁹ | 6 × 10⁹ | 5 × 10⁸ | 5 × 10⁸ | 9 × 10⁷ | 5 × 10⁷ | 1 × 10⁷ | 1 × 10⁷ |
| 4 | 6 × 10¹⁰ | 5 × 10¹⁰ | 1 × 10¹⁰ | 4 × 10⁹ | 9 × 10⁸ | 4 × 10⁸ | 5 × 10⁸ | 4 × 10⁸ | 1 × 10⁸ | 1 × 10⁸ |
| 5 | 4 × 10¹⁰ | 1 × 10¹⁰ | 1 × 10¹⁰ | 3 × 10⁹ | 1 × 10⁹ | 6 × 10⁸ | 5 × 10⁸ | 5 × 10⁷ | 2 × 10⁷ | 1 × 10⁷ |
| 6 | 3 × 10¹⁰ | 1 × 10¹⁰ | 1 × 10¹⁰ | 2 × 10¹⁰ | 6 × 10⁹ | 3 × 10⁹ | 1 × 10⁹ | 1 × 10⁹ | 1 × 10⁹ | 1 × 10⁹ |

The formulations disclosed herein, or formulated product, can be used alone or in combination with one or more other components, such as growth promoting agents and/or anti-phytopathogenic agents in a tank mix or in a program (sequential application called rotation) with predetermined order and application interval during the growing season. When used in a combination with the above-mentioned products, at a concentration lower than recommended on the product label, the combined efficacy of the two or more products (one of which is the said formulation disclosed herein) may be in certain cases greater than the sum of each individual component's effect. Hence, the effect is enhanced by synergism between these two (or more) products, and the risk for the development of pesticide resistance among the plant pathogenic strains is reduced.

The formulations disclosed herein can also be used in combination with other anti-phytopathogenic agents, such as plant extracts, biopesticides, inorganic crop protectants (such as copper), or chemical fungicides or bactericides with either single site, multisite or unknown mode of action. As defined herein, an “anti-phytopathogenic agent” is an agent that modulates the growth of a plant pathogen, particularly a pathogen causing soil-borne disease on a plant, or alternatively prevents infection of a plant by a plant pathogen. A plant pathogen includes but is not limited to a fungus, bacteria, actinomycete or virus.

The formulation can be applied by root dip at transplanting, specifically by treating a fruit or vegetable with the formulation by dipping roots of the fruit or vegetable in a suspension of said formulation (about 0.25 to about 1.5% and more particularly about 0.5% to about 1.0% by volume) prior to transplanting the fruit or vegetable into the soil.

The formulation can be added as an in-furrow application. Alternatively, the formulation can be applied by drip or other irrigation system. Specifically, the formulation can be injected into a drip irrigation system. In a particular embodiment, the formulation is applied at a minimum concentration of colony-forming units 10⁸-10⁹ CFU/ml in a volume of approximately quarts per acre. The spray concentration (after dilution with water-10 ml/L dilution) during field application will be minimum 10⁵-10⁶ CFU/ml.

The various embodiments of the present invention may achieve one or more of the following advantages and/or objects:

Firstly, the effective microbial isolates of the present invention are not only effective to control diseases such as late blight of potato but are also effective in controlling wilt caused by Fusarium sp., damping off caused by Pythium sp., Charcoal rot caused by Macrophomina sp., Powdery mildews, Downy mildews, Rusts, Smuts, Leaf blights, Leaf spots, Fruit rots, Die back and others.

Secondly, the formulations also function as soil enrichment solutions stimulating plant growth, rejuvenating the soil, and promoting the growth of beneficial soil microorganisms. The formulations contain microorganism spores and/or colonies that remain viable for at least about four months when stored at room temperature. The formulations provide soil enrichment solutions containing viable microorganism spores and/or colonies, particularly those useful for enriching poor, disturbed soils or soils having little or no microbial activity because of the heavy past use of chemicals and/or fertilizers.

The above description is for the purpose of illustrating and not limiting the present invention and teaching the person of ordinary skill in the art how to practice the invention. It is not intended to detail all those obvious modifications and variations of it which will become apparent to the skilled worker upon reading the description. It is intended, however, that all such obvious modifications and variations be included within the scope of the present invention as defined in the appended claims. The claims are meant to cover the claimed components and steps in any sequence which is effective to meet the objectives there intended unless the context specifically indicates the contrary.

1. A bio-fungicide formulation comprising a biologically pure culture of Bacillus amyloliquefaciens (MTCC 25554) and excipients, wherein the Bacillus amyloliquefaciens (MTCC 25554) is present in a range from 1 to 98% (wt./wt.).

2. A bio-fungicide formulation comprising a biologically pure culture of Bacillus subtilis subsp. spizizenii (MTCC 25552) and excipients, wherein the Bacillus subtilis subsp. spizizenii (MTCC 25552) is present in a range from 1 to 98% (wt./wt.).

3. A bio-fungicide formulation comprising a biologically pure culture of Bacillus amyloliquefaciens (MTCC 25555) and excipients, wherein the Bacillus amyloliquefaciens (MTCC 25555) is present in a range from 1 to 98% (wt./wt.).

4. A bio-fungicide formulation comprising a biologically pure culture of Bacillus velezensis (MTCC 25553) and excipients, wherein the Bacillus velezensis (MTCC 25553) is present in a range from 1 to 98% (wt./wt.).

5. A bio-fungicide formulation comprising a biologically pure culture of Bacillus safensis subsp. safensis (MTCC 25556) and excipients, wherein the Bacillus safensis subsp. safensis (MTCC 25556) is present in a range from 1 to 98% (wt./wt.).

6. A bio-fungicide formulation comprising a biologically pure culture of Bacillus spp. and excipients, wherein the Bacillus spp. is selected from the group consisting of Bacillus amyloliquefaciens (MTCC 25554), Bacillus subtilis subsp. spizizenii (MTCC 25552), Bacillus amyloliquefaciens (MTCC 25555), Bacillus velezensis (MTCC 25553), Bacillus safensis subsp. safensis (MTCC 25556), and a combination thereof, wherein the Bacillus spp. is present in a range from 1 to 98% (wt./wt.).

7. The bio-fungicide formulation as claimed in claim 1, wherein the excipient is selected from a surfactant, a encapsulant, an adjuvant, a sticking agents, a preservative, and water.

8. The bio-fungicide formulation as claimed in claim 7, wherein the surfactant is selected from the group consisting of Polyethylene glycol (PEG)-100, PEG-200, PEG-300, PEG-400, PEG-8000, PEG-20000, Tween-20, Tween-40, Tween-60, Tween-80, Span-20, Span-40, PEG 6000, glycerol, PVP K30, PEG 400, Tween 85, and a combination thereof, wherein the surfactant is present in a range from 0.001 to 31% (wt./wt.).

9. The bio-fungicide formulation as claimed in claim 7, wherein the encapsulant is selected from the group consisting of glycerol, PEG (Polyethylene glycol) and PVP (Polyvinylpyrrolidone), preferably glycerol present in a range from 2 to 5% (wt./wt.).

10. The bio-fungicide formulation as claimed in claim 7, wherein the sticking agent is xanthan gum, 0.4% xanthan gum, guar gum, gum acacia, Arabic gum, malto dextrin, starch soluble and insoluble, carboxy methyl cellulose or combination thereof, preferably, xanthan gum present in a range from 0.05 to 82% (wt./wt.).

11. The bio-fungicide formulation as claimed in claim 7, wherein the adjuvant is selected from the group consisting of olive oil, cashew oil, castor oil, sunflower oil, Pongamia oil, sesame oil, linseed oil, rice bran oil, ground nut oil, and a combination thereof, preferably, linseed oil and sesame oil present in a range from 2 to 98% (wt./wt.).

12. The bio-fungicide formulation as claimed in claim 7, wherein the preservative is selected from the group consisting of citric acid, calcium propionate, copper sulphate, magnesium sulphate, boric acid, calcium benzoate, molybdenum, and a combination thereof, wherein the preservative is present in a range from 0.01 to 0.25% (wt./wt.).

13. The bio-fungicide formulation as claimed in claim 1, wherein the formulation is formulated as an aqueous suspension or emulsifiable concentrate.

14. The bio-fungicide formulation as claimed in claim 1, wherein the fungal infections are caused by Phytophthora infestans.

15. The bio-fungicide formulation as claimed in claim 1, wherein the formulation comprises a chemical or biological pesticide.
